(19) United States
(12) Patent Application Publication
(10) Pub. No.: US 2025/0161646 A1
(43) Pub. Date: May 22, 2025

(54) A WEARBLE MICRONEEDLE SPRING APPLICATOR

(71) Applicant: Chee Yen LIM, Singapore (SG)

(72) Inventor: Chee Yen LIM, Singapore (SG)

(21) Appl. No.: 18/839,449

(22) PCT Filed: Mar. 31, 2022
(86) PCT No.: PCT/SG2022/050185

§ 371 Date: Aug. 19, 2024

(51) Int. Cl. A61M 37/00

(52) U.S. Cl. CPC A61M 37/0015; A61M 2037/0023

(57) ABSTRACT

In the first preferred embodiment (please refer to FIGS. 1-4), a low-profiled, wearable microneedle spring applicator (200) is provided. The applicator (200) comprises (a) a reusable skin tensioning and fixating device (220) which is made up of a skin tensioning base (240) and a strap (260); and (b) a disposable microneedle spring applicator (280) which is attachable to the skin tensioning base (240). In this preferred embodiment of the present invention, we use the compression force generated from fastening the strap (260) around a body to generate the holding force.

### FIELD OF THE INVENTION

The present invention relates to a wearable device for applying microneedle patches on the skin for drug delivery purposes. More particularly, the present invention relates to a wearable microneedle spring applicator which provides a continuous holding force for skin tensioning and fixation and holding microneedles in the skin (application pressure).

### BACKGROUND OF THE INVENTION

Microneedles are very effective in delivering drugs to the skin painlessly. The dissolvable microneedles has become very popular in recent years because of their ability to precisely deliver active ingredients to the targeted depth of the skin, painlessly. Since most biologics are degradable when administered through oral administration, dissolvable microneedles provide an attractive administration route to avoid the gastro-intestinal tract. These dissolvable microneedles are made of dissolvable matrix materials such as hyaluronic acid (HA), poly (vinylpolypyrrolidone) (PVP), carboxylmethyl cellulose (CMC), polyvinyl alcohol (PVA), and so on. Pharmaceutical active ingredients can be loaded in the dissolvable microneedle patches and be administered to the skin through the dissolution of the microneedles. For delivery of drug to take place successfully, these dissolvable microneedles first have to penetrate the skin and then dissolve by remaining in the skin for a length of time, for example a few minutes to a few hours.

As mentioned previously, there are two prerequisites for successful drug delivery by dissolvable microneedles: (1) the microneedles have to penetrate the skin, and (2) the microneedles have to remain in the skin for the dissolution to take place. Since these dissolvable microneedles are made of polymers that dissolve, naturally their physical strength and hardness are too low to cause skin penetration. The viscoelasticity and deformability of the skin make skin penetration by dissolvable microneedles even harder. Through our research, we discovered that when these dissolvable microneedles are applied to the skin at high speed, i.e., more than 1 m/s, or between 1-10 m/s, they are capable of penetrating the skin effectively. We hypothesize that under high application speed, the viscoelasticity of the skin does not have time to react to the penetration, causing the skin to be ‘frozen’ and be particularly vulnerable for the dissolvable microneedle to penetrate.

Separately, we also discovered that when the skin site is tensioned by disposing a tensioning hole on the skin site, the skin penetration of dissolvable microneedles become much reliable and consistent. This is due to the fact that the skin tensioning hole fixated and tension the skin so that it remains still and rigid when microneedles are inserted into the skin.

Once penetrated, to keep the dissolvable microneedles in the skin, a continual application pressure must be applied. If the continual application pressure is absent, the penetrated microneedles would be pushed out of the skin by the natural elastic behavior of the skin. Depending on the volume and the matrix material of the dissolvable microneedles, they take 3-30 minutes to dissolve, during which the continual application pressure must be applied. Apparently, this continual pressure requirement causes some inconvenience to the user.

WO2015/122838A1 filed by Lim described a method involving (a) tensioning and fixating the skin, (b) applying the microneedles on tensioned skin at high speed, and (c) continuously holding the microneedles against the skin while they are dissolving in the skin. However, this prior art requires the user to continuously hold the spring applicator and press it against the skin to provide the continual application pressure and skin tensioning force for a length of time, which may be proven to be inconvenient or impractical for the user if the application time is 30 minutes or longer. Such approach is only feasible when the holding time is short, for example a few seconds. Therefore, a device which provides the above functions but does not require the user's pressing of the microneedle patch is highly desirable. Moreover, the elongated shape of the prior art does not provide an ergonomic design for it to be worn on the skin.

There are currently several prior art devices which rely on adhesives for holding the microneedle patches on the skin. For example, U.S. Pat. No. 8,267,889B2 and U.S. Pat. No. 8,758,298B2 awarded to 3M provide a low-profile application device using a flexible membrane to actuate the microneedles by a normal or horizontal momentary force. Prior art WO2016/017561A1 filed by Zosano Pharma discloses a device for impacting microneedles on stratum corneum. A momentary force is applied to release a folding diaphragm which abruptly actuates the microneedles. these three prior arts employ the high-speed application, which is provided by the folding diaphragm/flexible membrane but lack the other two requirements (application pressure and skin tensioning force). U.S. Pat. No. 6,780,171B2 awarded to BD discloses an impact applicator with strap for coupling the applicator to a wrist or arm. The device applies a continuous pressure on a liquid reservoir for dispensing liquid into the skin via hollow microneedles. Prior art U.S. Pat. No. 8,579,862B2 awarded to MedRx discloses a concave applicator which when pressed against the skin raises the skin to momentarily touch a microneedle patch such that the microneedle patch will be affixed on and transferred to the skin via adhesive tape. It is worth noting that using adhesive means to hold the microneedles on the skin does not provide any continual application pressure. The adhesive force is to hold the microneedle patch on the skin and it arises when there is a detaching force. The application pressure has to be exerted externally or an innovative method is required to generate the continual application pressure using adhesive forces. (more explanation on adhesive forces in the second preferred embodiment's section)

Prior arts above use impact and high speed to apply microneedles on the skin. Other prior arts, such as WO2019/146618A1 to Cosmed focuses on fixating the skin using a ring on a strap. U.S. Pat. No. 11,116,953B2 to Medrx discloses a microneedle applicator with moving arms which pushes the skin outward to tension the skin. All the above prior arts emphasize on applying the microneedle patches using high speed and rely on adhesives to hold the patch on the skin. These prior arts do not aim to provide all the three functionalities described in prior art WO2015/122838A1 by Lim. For example, there is either no skin tensioning or fixating before applying the microneedle patch, or there is no continuous application pressure to push the microneedles against the skin to prevent them from dislodging from the skin.

Continuous application pressure is essential in keeping the microneedles in the skin to dissolve and release the active ingredients in the skin. In the absence of this continuous application pressure, the elasticity of the skin will push out the microneedles, causing the dissolving process to be disrupted or incomplete.

Prior art US2017/0050010A1 filed by Georgia Tech provides a microneedle applicator which is essentially using a thumb to press on the microneedle patch for 20 minutes. This prior art applies and continuously holds a microneedle patch against the skin without first tensioning the skin. In addition, as mentioned previously, asking the user to press the microneedle patch for 20 minutes may cause the user to abandon halfway due to lack of patience or soreness on the thumb. This prior art also lacks the high-speed application and skin tensioning requirements for effective microneedle's drug administration.

It is apparent that a wearable microneedle spring applicator, which provides all the essential functionalities of (a) tensioning and fixating an application skin site, (b) applying microneedles with high speed on the tensioned application skin site, and (c) continuously holding the microneedle patch against the tensioned application skin site for a length of time without user's intervention, is highly sought after. In our context, the user can be the person administering the microneedles or the patient receiving the microneedles.

### SUMMARY OF THE INVENTION

The present invention relates to a low profiled, wearable microneedle spring applicator (200) which provides several functionalities, namely (a) tensioning and fixating an application skin site, (b) applying microneedles with high speed on the tensioned application skin site, and (c) continuous holding the microneedles on the tensioned application skin site for a length of time without the user's intervention. The present invention discloses a wearable device that comprises a microneedle spring applicator, a fastening means to fasten the microneedle spring applicator to a skin site, and a skin tensioning hole to tension and fixate the skin for effective skin penetration. There are two preferred embodiments for the present invention, the first one is based on a strap approach and the second one is based on adhesive-tape approach.

In the first preferred embodiment (please refer to FIGS. 1-4), a low-profiled, wearable microneedle spring applicator (200) is provided. The applicator (200) comprises (a) a reusable skin tensioning and fixating device (220) which is made up of a skin tensioning base (240) and a strap (260); and (b) a disposable microneedle spring applicator (280) which is attachable to the skin tensioning base (240). The holding force in WO2015/122838A1 by Lim is provided normally by the user. In this preferred embodiment of the present invention, we use the compression force generated from fastening the strap (260) around a body to generate the holding force.

In the second preferred embodiment (please refer to FIGS. 7-8), a low-profiled, disposable wearable microneedle applicator (300) is provided. The disposable wearable microneedle spring applicator (300) comprises a bottom of the casing (340), a flange (320), an adhesive tape (360) and a bottom circular hole (380). The flange (320) is disposed around the bottom of the casing (340), and the adhesive tape (360) is placed on top of the flange (320), not below. It is worth noting that the adhesive tape (360) fastens the disposable wearable microneedle spring applicator (300) to the skin by sandwiching the flange (320) between itself and the skin. This is the only effective way to provide a continuous holding force to the wearable microneedle spring applicator (300). This is because by doing so, the flange (320) becomes the additional rigid space which is plunged into the skin thereby increasing the skin surface area (i.e. skin tensioning) and reducing the volume the skin (i.e. compression pressure or skin fixation). In WO2015/122838A1 by Lim, the holding force is provided vertically downward or normally by the user, but in this second preferred embodiment of the present invention, the compression force generated by the adhesive tape (330) when affixed on the tightened skin.

### BRIEF DESCRIPTION OF THE FIGURES

FIG. 1 shows the schematic diagram of the operation of a spring applicator

FIG. 2 shows a perspective view of the first preferred embodiment of the present invention

FIG. 3A shows the cross-sectional view of the first preferred embodiment

FIG. 3B shows the cross-sectioned exploded view of the first preferred embodiment

FIG. 4 shows the perspective view of the disposable microneedle spring applicator (280)

FIG. 5A shows the compression force distribution exerted by the strap (260) around a body

FIG. 5B shows the bulging skin surface caused by the bottom circular hole (240) and the strap (260)

FIG. 6A shows a perspective view of the skin tensioning base (240) which is incorporated with a compression ring (249)

FIG. 6B shows a cross-sectional view of the skin tensioning base (240) which is incorporated with a compression ring (249)

FIG. 7 shows a variant of the first preferred embodiment which comprises a disposable microneedle spring applicator and a snap-on strap

FIG. 8A shows a perspective view of the second preferred embodiment

FIG. 8B shows an exploded view of the second preferred embodiment

FIG. 8C shows a close-up bottom view of the second preferred embodiment

FIG. 9 shows a variant of the second preferred embodiment with annular adhesive tape

FIG. 10A shows the sandwiched position of the adhesive tape when a prior art device is used

FIG. 10B shows the position of the adhesive tape when the second preferred embodiment is applied

### DETAILED DESCRIPTION OF THE INVENTION

Most microneedles require an applicator to apply the microneedles to the skin effectively. According to WO2015/122838A1 filed by Lim, the three requirements for effective skin application by microneedles are: (a) tensioning and fixating the skin, (b) applying the microneedles on tensioned skin at high speed, and (c) continuously holding the microneedles against the skin normally (i.e., in a perpendicular direction the skin) while they are dissolving in the skin. According to our research and development, we discover that the dissolving time of microneedles may take 3-30 minutes depending on the size of microneedles if a microneedle application is 30 minutes, requiring the user to continuously press the microneedles against the skin will be too demanding; the user may give up halfway due to fatigue, or the direction (i.e. not normally or not perpendicularly to the skin) and magnitude of the pressing force may be changed over time, seriously affecting the drug delivery outcome. Hence, the third requirement above by Lim (WO2015/122838A1) requires one more condition, in which the continuous holding of microneedles is achieved by an independent external normal force without the intervention of the user. In this context, normal means perpendicular to the skin. The present invention discloses a wearable microneedle spring applicator that (a) first tensions and fixates the skin, (b) then applies microneedles at high speed, (c) and finally provides a continuous force for holding the microneedles without the intervention from the user.

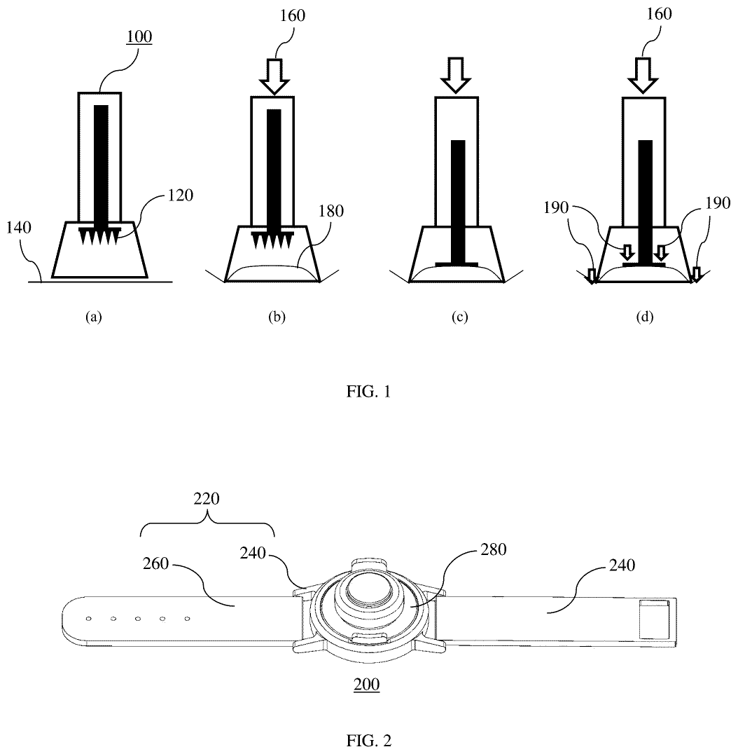

FIG. 1 shows a schematic diagram for the operations of a typical handheld spring applicator when it is used to apply a microneedle patch onto the skin surface. Normal spring applicator requires a compression spring to work. There is a compression spring disposed in the microneedle applicator (100), but it is not shown in the figure to present a simplified and clear diagram. FIG. 1 (a) show a handheld spring applicator (100) loaded with a microneedle patch (120) before touching the skin surface (140). In FIG. 1 (b), the spring applicator (100) is exerted with an external force (160) causing a compression onto the skin surface (140), the skin will be fixated and tensioned to form a bulging skin surface (180). Subsequently, in FIG. 1 (c) the spring applicator (100) is triggered, and the microneedle patch (120) is applied and inserted into the bulging skin surface (180). FIG. 1 (d) shows force distribution, the external force (160) exerted on the spring applicator (100) is distributed to skin (190) to hold the microneedles in the skin and to fixate and tension the bulging skin surface (180). It is important to note that the arrow representing the external force (160) is a general representation only, in real life, external force can be exerted at anywhere on the spring applicator and the exerted force will be re-distributed to the skin surface to hold the microneedles in the skin and to tension and fixate the skin.

The following preferred embodiments of the present invention demonstrate how the continuous holding force exertion can be maintained without requiring the user's intervention. The first preferred embodiment is suitable for long duration, it is used when the microneedle application time is more than 10 minutes to 12 hours. On the other hand, the second preferred embodiment is suitable for short duration application, it is used when the microneedle application time is less than 10 minutes.

### The First Preferred Embodiment

FIG. 2 shows a perspective view of the preferred embodiment of the present invention. In the preferred embodiment, a low-profiled, wearable microneedle spring applicator (200) is provided. This low-profiled, wearable microneedle spring applicator (200) comprises (a) a reusable skin tensioning and fixating device (220), which is furhter made up by a skin tensioning base (240) and a strap (260); and (b) a disposable microneedle spring applicator (280) that is attachable to the skin tensioning base (240). The disposable microneedle spring applicator (280) is operated by a compression spring or any other potential energy storing device which actuates a microneedle patch to impact the skin upon triggering. A person skilled in the art should reasonably understand the details of the basic operations of a spring applicator, so the details are not included herein.

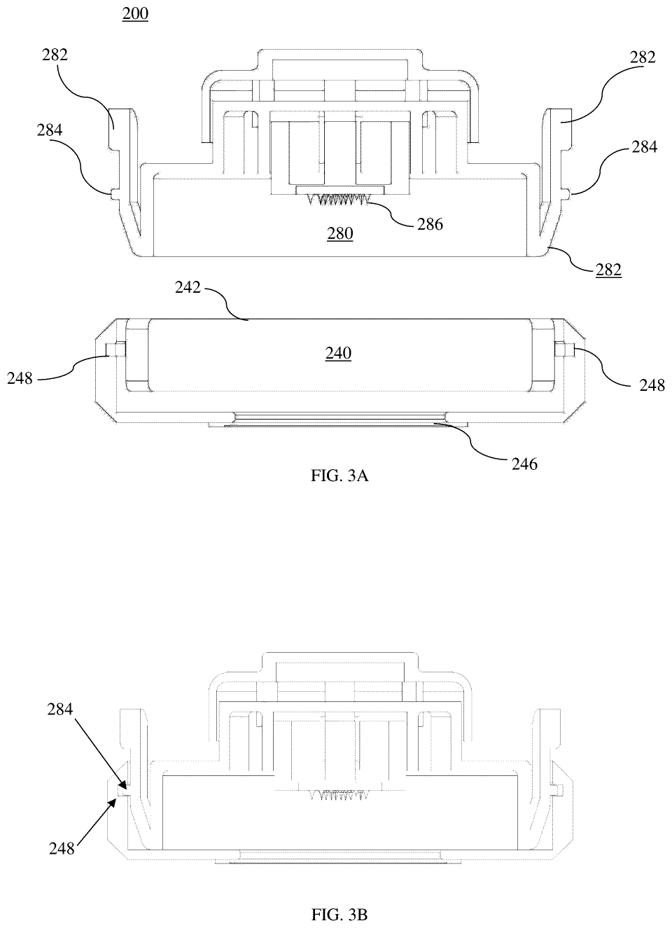

FIGS. 3A and 3B show the cross-sectioned diagrams of the skin tensioning and fixating device (220) in the first preferred embodiment. This is the key component that provides, when strapped on to the skin, the holding force (160) in FIG. 1 which requires no intervention from the user. This device (220) further comprises a skin tensioning base (240) which further comprises a top open face (242) and a bottom circular hole (246). The top open face (242) is to receive the disposable microneedle spring applicator (280) while the bottom circular hole (246) acts like a skin tensioning hole which tensions and fixates the skin when strapped on to the skin.

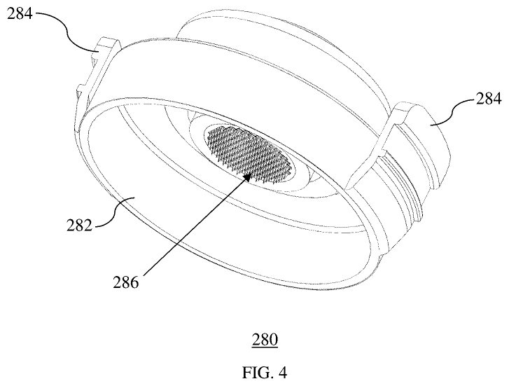

In FIG. 4, the disposable microneedle spring applicator (280) comprises a casing (282) which is disposed with at least a latching valve (284), a compression spring (not shown for simplified presentation) and a microneedle patch (286). The latching valves (284) will engage with the latching feature (248) via the top open face (242) of the skin tensioning base (240). The latching valves (284) are elastic and compressible to allow engagement and disengagement with the latching feature (248) on the skin tensioning base (240). During the engagement step, the latching valves (284) are momentarily compressed, and the latching valves will engage and lock into the latching feature (248). During the disengagement step, the latching valves (284) will again be momentarily compressed, and they will dislodge from the latching feature (128), allowing the disposable microneedle spring applicator (280) to detach from the skin tensioning base (240).

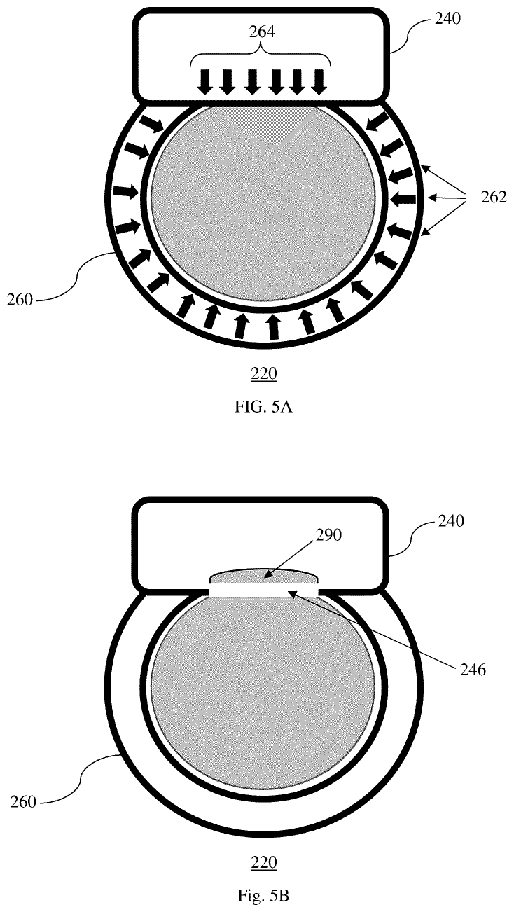

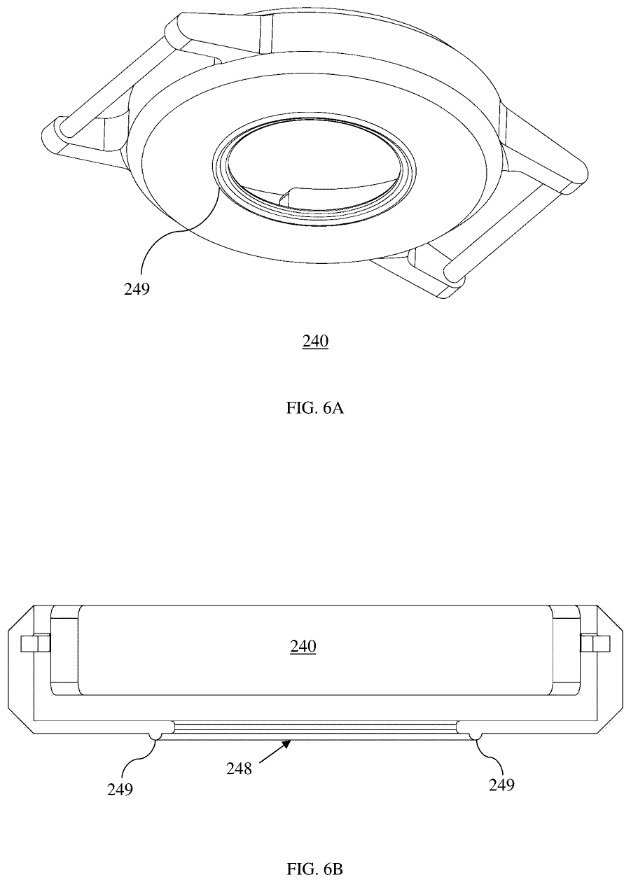

FIG. 5A shows the compression force distribution exerted by the strap (260) when it is fastened and tightened around a body. The compression force is represented by the arrows (262) around the strap (260) and the arrows (264) on the skin tensioning base (240). Both compression forces (262) and (264) come from the pressure the strap (260) generates. When the bottom circular hole (246) is pressed into the skin due to the fastening of the strap (260) on the skin, the compression force (264) provides the holding force around the bottom circular hole (246) which subsequently tensions and fixates the skin, forming a bulging skin surface (290) for the subsequent microneedle's penetration, as shown in FIG. 5B. After microneedles' penetration, compression force (264) also provides the holding force to keep microneedles in the skin. A protruding ring (249) may be incorporated around the bottom circular hole (246) to enhance the skin tensioning and fixating effect, as shown in FIGS. 6A and 6B. The protruding ring (249) acts as an additional rigid volume that will be plunged into the skin thereby increasing the skin's surface area and reducing the skin's volume. By doing so, the skin tension and its compression pressure are increased by the protruding ring (249). (Please also see FIG. 9 and its explanation)

To generate the compression force, the strap (260) has to be fastened and tightened around a part of the body, e.g., a limp, the head, the torso, the back etc. The convenient places for applying the first preferred embodiment are wrists, arms, upper arms and legs. When the strap (260) is fastened and tightened around the body, the body is mildly compressed thereby providing a radial outward force, which in turn induces the compression force from the skin tensioning and fixating device (220). (Newton's 3rd Law of motion: action and reaction force are equal and opposite)

In the first preferred embodiment, the skin tensioning and fixating device (220) may comprise separately a skin tensioning base (240) and a strap (260), or may be integrally molded as one single component (the skin tensioning base (240) is integrally molded with the strap (260) and formed a single part). When made as separate components, the skin tensioning base (240) can be made of metals or common plastic materials such as PC, PE, ABS, etc. The top open face (242) of the skin tensioning base (240) may be disposed with latching feature (248) such as a groove such that the disposable microneedle applicator (280) can be firmly attached to the skin tensioning base (240) via engaging or latching to the latching feature (248). This latching feature (248) may not be limited to a groove or a plurality of grooves, it can be in the form of hooks, indents, tabs, so on and so forth which are known to a person skilled in the art.

The strap (260) in the first preferred embodiment can be elastic or rigid. A common material for making this strap (260) is silicone rubber, leather, and other fabric materials. Other mouldable elastic materials such as TPE (thermoplastic elastomer) or TPU (thermoplastic polyurethane) are suitable candidates for making the strap (260). In addition, the strap (260) may be made of fabric or other non-elastic materials too. Particularly, the strap (260) may be made of adhesive tape. The disposable microneedle spring applicator (280) in the first preferred embodiment is made of plastic materials such as ABS, PC or PS, or a blend material known to any person skilled in the art. It is obvious to the persons skilled in the art that latching mechanism in the first preferred embodiment is not limited to the present disclosure but includes all common mechanisms for engaging mechanical parts.

When in operation, the user will first fasten and tighten the strap (260) around a skin site such as a wrist, so that the skin tensioning base (240) will exert a compression force towards the skin, causing the skin site to be tensioned and fixated within the bottom circular hole (246). A bulging skin surface will then form. Then, the user will mount the disposable microneedle spring applicator (280) on the skin tensioning base (240) by slightly compressing the latching valves (282). Next, the user may remove a safety pin (not shown in FIGS. 3 and 4) of the disposable microneedle spring applicator (280) and trigger a compressed spring to propel a microneedle patch at high speed (1-10 m/s) to impact the skin. The high-speed impact enables the microneedles to penetrate the skin. Once the microneedles are in the skin, they are held in the skin by the compression force (264) generated by the straps (260) and dissolve rapidly (e.g. 10 seconds to 10 minutes) in the skin to release the drugs. The user will continue to wear the straps (260) for a length of time until the specified drug delivery duration is achieved. The first embodiment is designed to target long application time (10 minutes to 12 hours).

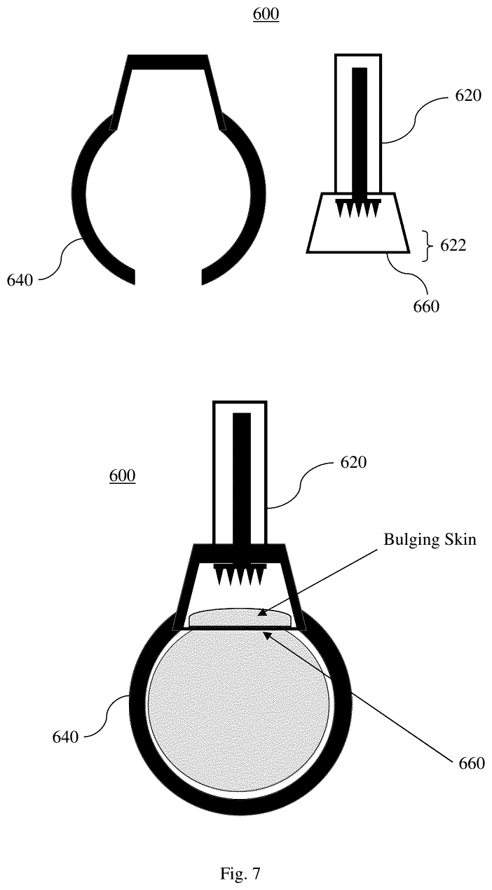

There is more than one embodiment which can represent the present invention. For example, as shown in FIG. 7, a variant of first embodiment is disclosed. This variant (600) comprises a microneedle spring applicator (620) having a distal end (622), a snap-on strap (640) and a skin tensioning hole (660) which is disposed at the distal end (622) of the microneedle spring applicator (620). When in use, the snap-on strap (640) will be snapped on to the microneedle spring applicator (620) and then fastened around a body in a way which the skin tensioning hole (660) is compressed against the skin. Once the microneedle spring applicator is fastened to the skin, the microneedles will be actuated to apply on the skin. The fastening should be tight enough to generate compression pressure (264) as shown in FIG. 5A. The snap-on strap (640) will fasten the microneedle spring applicator (620) securely on the skin and provide the required holding force for (a) holding the microneedles in the skin, (b) tensioning and fixating the skin via the skin tensioning hole (660), and (c) overcoming the impact due to application of microneedles.

### The Second Preferred Embodiment

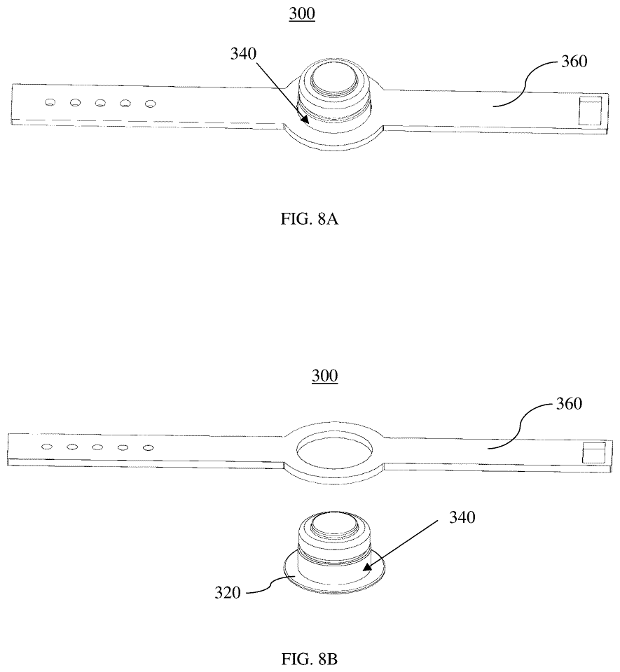

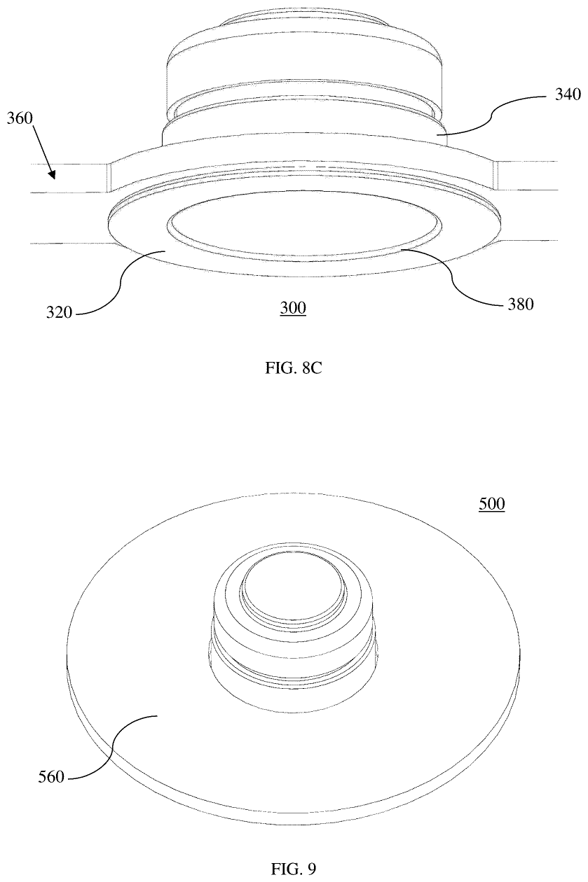

In the first preferred embodiment of the present invention, it comprises two components: (a) the skin tensioning and fixating device (220), which is reusable, and (b) disposable the microneedle spring applicator (280), which is one-time use. In this second preferred embodiment of the present invention, the entire wearable microneedle applicator device will be made disposable by incorporating two adaptations to the first preferred embodiment. FIGS. 8A-8C show respectively the perspective view, the exploded view and the close-up view of the new disposable wearable microneedle spring applicator (300). The first adaptation is the removal of the latching valves (248 in the first embodiment) and the addition of a surrounding flange (320) around the bottom of the casing (340). The second adaptation is the replacement of the skin tensioning and fixating device (220 in the first embodiment) with a ring-shape adhesive tape (360).

In FIG. 8C, the bottom view of the disposable wearable microneedle spring applicator (300) is shown. The adhesive tape (360) is placed on top of the flange (320), not below. It is worth noting that the adhesive tape (360) fastens the disposable wearable microneedle spring applicator (300) by sandwiching the flange (320) between itself and the skin. This is the only effective way to provide a continuous holding force to the wearable microneedle spring applicator (300). This is because by doing so, the flange (320) becomes the additional rigid space which is plunged into the skin thereby increasing the skin surface area (i.e. skin tensioning) and reducing the volume the skin (i.e. compression pressure or skin fixation). By varying the volume of the flange (320), e.g. its thickness or size, the desirable compression pressure and skin tension can be achieved. Then, the area of the adhesive tape can be determined to provide adequate adhesion force to hold the wearable device (300) on the skin with the desired compression pressure. The adhesive force should be at least equal or more than the sum of the required stretching force, the required compressive force and the impact force of the microneedles to be effective. A variant of the disposable wearable microneedle spring applicator (500) is disclosed in FIG. 9. The device (500) comprises a substantially round adhesive tape (560) that is disposed on top of the flange (not shown in the figure).

Although there is no protruding ring disposed on the device, a person skilled in the art can readily add a protruding ring or any rigid features around the bottom circular hole (380) to enhance the skin tensioning and fixating effect. With additional protruding ring around the bottom circular hole (380) will further enhance the compression pressure, but this will also increase the required adhesive force, so a larger adhesive tape is required to adhere the wearable microneedle spring applicator (300).

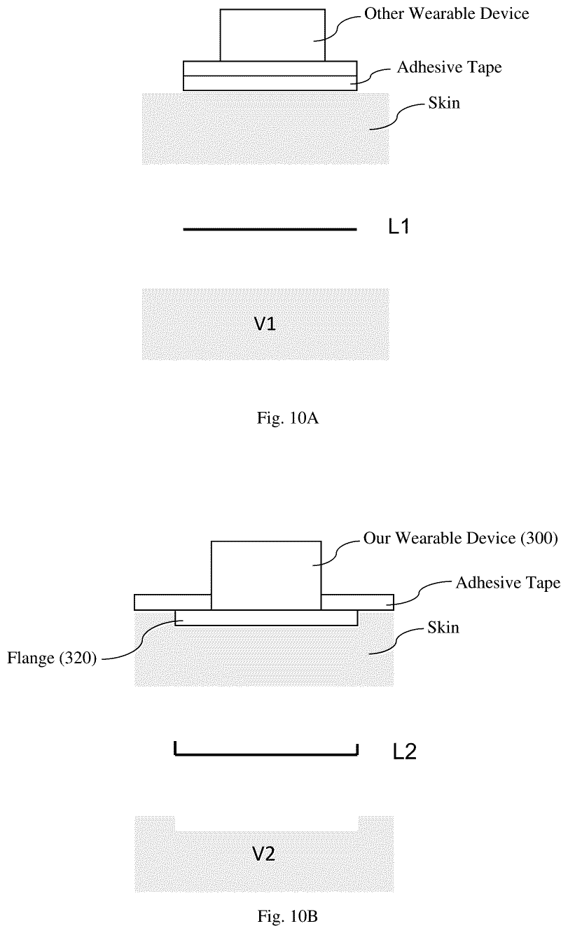

It is worth pointing out that common wearable devices including wearable microneedle applicators in the prior arts have the adhesive tape disposed between the device and the skin, as shown in FIG. 10A. The adhesion of the device on the skin is sufficient to stick the device on the skin, but it does not provide any compression pressure or stretching force to the skin because the skin's surface area (L1) is not increased and the skin does not reduce in volume (V1). In the contrary, as shown in FIG. 10B, the second preferred embodiment of the present invention introduces a solid space (400) (i.e. the flange (320) between the adhesive tape and the skin) which causes the skin to stretch by increasing the surface area (L2) and exerts compression pressure to the skin by forcing the skin to reduce its volume (V2) to make room for the flange (320). By comparing the cross-sectional skin surfaces between the common wearable device in FIG. 10A and that of the present invention in FIG. 10B, it is obvious that the present invention has longer (or larger) skin surface area (L2>L1) and lesser skin volume (V2<V1), a direct indication that the present invention can provide continuous holding force for maintaining the skin tensioning and fixation, as well as for holding the microneedles in the skin to dissolve once they are inserted into the skin.

When in operation, a user will remove the paper liners from the adhesive tape (360) and affix the adhesive tape (360) on the skin. When the wearable microneedle spring applicator (300) was adhered on a skin site with adhesive tape (360), the flange (320), which is plunged into the skin, causes the skin to increase in surface area and to reduce in volume, thereby tensioning and fixating the skin for skin penetration by microneedles. the adhesion due to the adhesive tapes will continue to hold the flange (320) in the skin to provide the holding force required to hold the microneedles in the skin to dissolve and deliver the drug completely. The second preferred embodiment is designed for short application time, e.g. 10 minutes or less.

1. A wearable microneedle spring applicator, comprising
(a) a microneedle spring applicator having a distal end,
(b) a skin tensioning hole, and
(c) a skin fastening means which fastens the microneedle spring applicator to a skin site while generating compression pressure to the skin site.

2. A wearable microneedle spring applicator in claim 1, wherein the skin tensioning hole is formed by the distal end of the microneedle spring applicator.

3. A wearable microneedle spring applicator in claim 1, wherein the skin fastening means has a snap-on design to secure the microneedle spring applicator at the skin site.

4. A wearable microneedle spring applicator in claim 1 wherein the skin fastening means is a strap.

5. A wearable microneedle spring applicator in claim 1 wherein the skin fastening means is made of adhesive tape.

6. A wearable microneedle spring applicator in claim 1 providing a holding force at least equal to or more than the total force required for:
(a) tensioning and compressing the targeted skin site,
(b) holding the microneedles in the skin to dissolve, and
(c) overcoming the impact force during application of microneedles.

7. A wearable microneedle spring applicator, comprising
(a) a skin tensioning base, which comprises a top open surface and a bottom circular hole,
(b) a strap, and
(c) a disposable microneedle spring applicator,
Wherein the disposable microneedle spring applicator is attachable to the skin tensioning base through the top open surface.

8. A wearable microneedle spring applicator in claim 7, further comprising a protruding ring which is disposed around the bottom circular hole on the skin tensioning base.

9. A wearable microneedle spring applicator in claim 7, in which
(a) the disposable microneedle spring applicator comprises at least one latching valve,
(b) the skin tensioning base comprises at least one latching feature,
Wherein the latching valve will engage with the latching feature when the disposable microneedle spring applicator is attached to the skin tensioning base.

10. A wearable microneedle spring applicator in claim 7 providing a holding force at least equal to or more than the total force required for:
(a) tensioning and compressing the targeted skin site,
(b) holding the microneedles in the skin to dissolve, and
(c) overcoming the impact force during application of microneedles.

11. A disposable wearable microneedle spring applicator, comprising
(a) a microneedle spring applicator comprising a bottom of the casing,
(b) a bottom circular hole which is disposed at the bottom of the casing,
(c) a flange which is disposed at the bottom of the casing, and
(d) an adhesive tape which is disposed on top of the flange such that when applied to the skin, the adhesive tape sandwiches the flange between itself and the skin.

12. A disposable wearable microneedle spring applicator in claim 11, further comprising a protruding ring which is disposed around the bottom circular hole.

13. A disposable wearable microneedle spring applicator in claim 11, wherein the flange is sandwiched between the adhesive tape and the skin is plunged into the skin, causing the skin to increase its surface area and reduce its volume, thereby increasing the skin tension and the compression pressure at the skin site.

14. A disposable wearable microneedle spring applicator in claim 11 providing a holding force at least equal to or more than the total force required for:
(a) tensioning and compressing the targeted skin site,
(b) holding the microneedles in the skin to dissolve, and
(c) overcoming the impact force during application of microneedles.

15. A disposable wearable microneedle spring applicator in which the adhesive tape has a surface area that generates adhesion force mentioned in claim 14.
